(19) United States
(12) Patent Application Publication
(10) Pub. No.: US 2007/0010492 A1
(43) Pub. Date: Jan. 11, 2007

(54) Composition and method for reducing homocysteine caused by drugs containing methyl compounds

(76) Inventor: Robert James Generale, Yonkers, NY (US)

Correspondence Address: ROBERT J. GENERALE, 671 BRONX RIVER ROAD, YONKERS, NY, 10704, US

(21) Appl. No.: 11/482,557

(22) Filed: Jul. 7, 2006

Related U.S. Application Data
(60) Provisional application No. 60/697,524, filed on Jul. 11, 2005.

(51) Int. Cl. A61K 31/675; A61K 31/525; A61K 31/205; A61K 31/14

(52) U.S. Cl. 514/89; 514/251; 514/554; 514/642

(57) ABSTRACT

This invention relates to a method and composition of limiting homocysteine. The composition consists of a problematic drug which specifically contains at least one CH3 methyl group at one or more end(s) of its chemical structure. This problematic drug is combined in an embodiment with one or more antidotal cofactors from among: folic acid, vitamin B6, vitamin B12, trimethylglycine and choline.

### CROSS-REFERENCES TO RELATED APPLICATIONS

This application claims the benefit of provisional patent application Ser. No. 60/697,524, filed 2006 July 11 by the present inventor.

### FEDERALLY SPONSORED RESEARCH

Not Applicable

### SEQUENCE LISTING OR PROGRAM

Not Applicable

### BACKGROUND

1. Field of the Invention

This invention relates to both prescription and over the counter (OTC) drugs, specifically as it relates to its antidotal embodiment to reduce homocysteine.

2. Prior Art

Homocysteine is a toxic metabolite of the amino acid methionine.

Homocysteine has been linked to an elevated risk of cardiovascular disease and strokes.

Dr. Kilmer McCully, in 1969, was the first to suggest that high-normal levels of homocysteine constitute a risk factor for cardiovascular disease. Early support for this concept come from a study published in 1976 by Wilcken and Wilcken, who reported that following an oral dose of methionine, serum homocysteine levels tended to be higher in patients with premature coronary disease than in healthy controls. Dr. Meir Stampfer and his colleagues at Harvard, using data from the large Physicians Health Study, provided striking confirmation that the risk of myocardial infarction was threefold higher in subjects with homocysteine levels in the top 5% of values, compared to subjects with homocysteine in the bottom 90%.

The major problem with many of todays drugs, specifically all coxibs, most NSAIDs, SSRi's, fibrates, and anti-epileptic drugs among many other classes have been identified to cause adverse cardiovascular events. These events include heart attacks and strokes.

The reason for these adverse cardiovascular events is due to an increase in homocysteine from the use of these drugs. All the problematic drugs contain methyl compound(s) in their formulations. Specifically, these drugs contain at least one (1) methyl group (CH3) as a single bond molecule at one or more end(s) of their chemical structure. A CH3 molecule is made of one (1) carbon atom and three (3) hydrogen atoms. The use of these drugs combined with the lack of testing of patients homocysteine plasma levels, are causing these adverse cardiovascular events. The use of these drugs combined with low plasma levels of folic acid and/or vitamin B-6 and/or vitaminB-12 and/or trimethyl glycine (TMG), and/or choline cause cardiovascular adverse events.

The homocysteine problem has particular consequences in older patients taking the problematic drugs. This is because they do not normally maintan adequate levels of the antidotal co-factors.

Patients with rheumatoid arthritis have elevated homocysteine. Therefore it is of vital importance that their pain relief drugs contain the antidotal co-factor embodiment.

The following are examples of merely some of the drugs which have the chemical structure as previously described and have a record and/or side effects listed involving adverse cardiovascular events:

arcoxia, carbamazepine, celecoxib, evra, fibrates (class), gabitril, ibuprofen, imitrex, indomethacin, ketamine, lexapro, lotronex, lumiracoxib, metformin, methotrexate, methylphenidate, mobic, naproxen, parecoxib, pargluva, phenytoin, piroxicam, rofecoxib, sulindac, valdecoxib, zyprexa.

The following are examples of some of the drugs which do not have the chemical structure as previously described, and do not have a record and/or side effects involving adverse cardiovascular events:

aspirin, daypro, diclofenac, dolobid, dyazide, furasimide, hydrochlorothiamide, levoxyl, lisinipril, magnesium salicylate, orudis, synthroid, warfarin.

The pharmaceutical companies do not recognize the problem with homocysteine.

Universal testing for homocysteine is not performed by the medical community.

Pharmaceutical companies do not check a drug during development for its specific effect on plasma homocysteine.

Even through Phase IV clinical trials, testing is not performed for the plasma homocysteine levels of patients.

Unfortunately, the pharmaceutical companies have not made the necessary modifications to their drugs formulations to correct the problem with homocysteine.

My embodiment addresses this problem and contains the composition and method to control the homocysteine caused by problematic drugs.

### OBJECTS AND ADVANTAGES

Accordingly, several objects and advantages of my invention are as follows:

My embodiment allows the problematic drug to fullfil its original goal. This is fully retained white my embodiment provides the antidotal control of the homocysteine danger.

Decades of studies have proven the safety and efficacy of the antidotal co-factors to control homocysteine.

My invention is the first embodiment which allows the safe use of drugs manufactured with the problematic methyl compound(s).

My embodiment will allow for the safe use of such drugs, as designed, without the resulting dangers of homocysteine.

Other objects and advantages are:

The additional costs of this embodiment to achieve the resolution of homocysteine dangers are relatively modest.

Drugs containing methyl compound(s) can be used safely toward their intended goal.

Future drugs containing methyl compound(s) can continue to be developed, manufactured, and dispensed safely with the embodiment design of my invention.

The embodiment of this invention allows the manufacturer of the problematic drug(s) to retain control. This control will allow the manufacturer to ensure the proper dosing of the antidotal co-factor(s).

This invention contains the embodiment of the only known antidote(s) to safely control homocysteine.

Beyond making the drugs safer, there is an additional advantage of the embodiment. In the case of pain relief drugs, the embodiment will specifically extend the pain relief cycle by stabilizing methionine.

Further objects and advantages will become apparent from consideration of the ensuing description.

### SUMMARY

My invention includes a method and composition to reduce homocysteine created from the use of drugs containing methyl compounds. My invention is for an embodiment which includes the problematic drug with the homocysteine antidotal co-factor(s).

### DRAWINGS

Not applicable.

### DETAILED DESCRIPTION—PREFERRED EMBODIMENT

My invention is a composition containing a prescription or OTC drug, and one or more homocysteine antidotal co-factors in an embodiment. This embodiment is to be designed for oral administration as tablet, capsule, gelcap, or other suitable oral administration vehicle.

The drug used in this embodiment is one which contains one (1) or more methyl compounds). The homocysteine antidote in the embodiment includes the following co-factors: folic acid, and/or vitamin B6, and/or vitamin B12 and/or trimethylglycine (TMG) and/or choline. The antidotal co-factor embodiment is designed to reduce the homocysteine level which may otherwise be increased in a person taking the drug. The problematic drugs formulations can be as medical needs demand. The problematic drugs dosages can be as medical needs demand.

A baseline for the antidotal co-factors of the embodiment to be added to the drug formulation is:

at least 800 mcg Folic Acid (Folate) and/or

at least 100 mg Vitamin B-6 (preferably Pyridoxyl 5-Phosphate and/or at least 1000 mcg Vitamin B-12 (preferably Methylcobalamin) and/or

at least 500 mg Trimethylglycine (TMG) and/or

at least 500 mg Choline

The baseline is to be used by the pharmaceutical companies while plasma homocysteine levels are observed during development and clinical trials. Adjustments will be made based on the individual drug's potential to create homocysteine.

In all cases, a patient's individual physiology must be combined with plasma homocysteine testing to determine if any antidotal co-factors are to be administered to lower homocysteine to safe levels prior to the commencement of use of the problematic drug(s).

This danger of homocysteine relates primarily to problematic drugs taken for chronic conditions. This allows homocysteine more time to develop. In those cases where problematic drugs are taken for shorter periods, the homocysteine cannot be ignored. Its cardiovascular effects will develop dependent upon the drugs design and its homocysteine potential. But most importantly, its cardiovascular effects will depend on the antidotal co-factors available.

Operation

My invention contains the antidotal embodiment for adverse cardiovascular events linked to certain classes of, and individual prescription and OTC drugs.

My embodiment encompasses the solution by addressing the three factors relevant to the problem.

A problematic drug contains at least one methyl group (CH3) at one or more endis) of its chemical structure.

Concurrent with this is the users plasma level of specific antidotal co-factors. These co-factors include Folic Acid, Vitamin B6, Vitamin B12, Trimethylglycine and Choline.

The third factor of the problem is the users plasma level of homocysteine, previously described for its detrimental cardiovascular effects.

Extended pain relief is initiated from a sustained release of methionine. The process starts with a drug containing CH3 molecule(s), as described previously. When the drug is ingested, it is converted to methionine, an amino acid, which is able to cross the blood brain barrier. Once in the brain, the methionine stimulates the basal ganglion region which causes a release of dopamine and norepinephrine. This action was explained by Dr. Richard Ross while at UCLA.

Drugs with this chemical structure design are often used for their anti-inflammatory activity. The exact action of these drugs on prostaglandins for their anti-inflammatory is not fully understood.

When the antidotal co-factors are not adequate, the homocysteine begins to cascade since there is no control of it. Homocysteine is transportred through the vascular system by LDL cholesterol. Homocysteine eventually accumulates in the endothelial walls, causing intense inflammation, As the inflammation continues, the body attempts to repair the damage by aggregating cholesterol at the inflammed sites. This process eventually leads to clots, heart attacks and other thrombotic events.

Homocysteine, although always present in the body, should be kept well within recognized medical limits. Homocysteine, when in the presence of adequate antidotal co-factors, is transsulferated by Vitamin B6 to cysteine, a benign amino acid. And/or it is remethylated back to methionine by the other antidotal co-factors.

In transsulfuration, the vitamin B6 dependent enzyme, cystathionine b-synthase (CBS), irreversibly generates cystathionine from homocysteine. The rate at which the process takes place is determined by the habitual dietary intake of methionine and body levels of Vitamin B-6. When this delicate balance is upset by the introduction of methionine from drugs containing methyl compounds, the only way to minimize homocysteine is to maximize CBS. This is done by increasing the bodily intake of Vitamin B6, which converts the homocysteine into a harmless metabolite, cystathionine.

The remethylation process begins with the undesired metabolic conversion of methionine to homocysteine. The body can prevent this if there are enough available antidotal co-factors, particularly Vitamin B12 and Folic Acid. With the adequate presence of these and the other co-factors, there will be continuous conversion of homocysteine back to methionine and/or harmless metabolites

Homocysteine is a direct result of excess, unmetabolized methionine caused by inadequate antidotat co-factors. My embodiment provides for the antidotal co-factors to be combined with the problematic drugs.

### CONCLUSION, RAMIFICATIONS, AND SCOPE

The chemical design of drugs was born out of medical necessity. Isolated discoveries began to present the possibilities of specific dangers of these drugs.

The most important specific discovery was homocysteine.

My embodiment invention is the first to directly address the cause and effect between a drugs formulation and design and resulting adverse cardiovascular events. The CH3 location as described is present in vast numbers of drugs. There is an almost perfect correlation between the CH3 presence and locaton and resulting adverse cardiovascular events. Conversely, drugs which do not have that chemical structure do not have the same correlation to adverse cardiovascular events.

The link is unmetabolized and uncontrolled homocysteine. This is caused by the problematic methyl drugs and/or inadequate antidotal co-factors. The antidotal co-factors have been intensely researched. The antidotal co-factors named are the only possible control of homocysteine.

My embodiment of antidotal cofactors and problematic drugs must be used immediately. This will allow pharmaceutical companies to reformulate to correct the homocysteine dangers with the current problematic drugs. It will also allow future development of drug design without the deadly side effects of homocysteine and resulting adverse cardiovascular events.

### REFERENCES

Huemer M et al, “Hyperhomocysteinemia in children treated with antiepileptic drugs is normalized by folic acid supplementation” Epilepsia, October; 46(10):1477-83, 2005.
Olthof M R et al. “Choline supplemented as phosphatidylchorine decreases fasting and post-methionine loading plasma homocysteine concentrations in healthy men”, Am J Clin Nutr, July; 82(1): 111-7, 2005.
Unlu Y et al, “Hyperhomocystenoemia as a risk factor for deep-vein thrombosis”, European Journal of Vascular Endovascular Surgery September 30 (3):315-8, 2005.
Miggiono, G A et al, “Diet, nutrition and rheumatoid arthritis”. Clin Ter., May-June 156(3): 115-23, 2005.
Ditscheid B et al, “Effect of I-methionine supplementation on plasma homocysteine and other free amino acids: a placebo-controlled double-blind cross-over study”, Eur J Clin Nutr. June; 59(6): 768-75, 2005.
Quere I et al, “Homocysteine and venous thrombosis”, Semin Vasc Med, May 5(2) 183-9, 2005.
Dierkes J et al, “Effect of drugs on homocysteine concentrations”, Semin Vasc Med, May 5(2) 124-39, 2005,
Holm P I et al, “Betaine and folate status as cooperative determinants of plasma homocysteine in humans” Arterioscler Thromb Vasc Biol., February; 25(2): 379-85, 2005.
da Costa K A et al, “Choline deficiency in mice and humans is associated with increased plasma homocysteine concentration after a methionine load”, American Journal of Clinical Nutrition, February; 81(2): 440-4 2005.
McCully K S “Hyperhomocysteinemia and arteriosclerosis: Historical perspecfives” Clin Chem Lab Med. 43(10): 980-6, 2005
Riba R et al, “Altered platelet reactivity in peripheral vascular disease complicated with elevated plasma homocysteine levels”, Atherosclerosis, July; 175(1): 69-75, 2005.
Yagisawa M et al, “Effects of intravenous betaine on methionine-loading-induced plasma homocysteine elevation in rats”, J Nutr Biochem, November; 15(11): 666-71, 2004.
Dierkes J et al, “The effect of fibrates and other lipid-lowering drugs on plasma homocysteine levels” Expert Opin Drug Saf. March, 3(2): 101-11, 2004.
Ventura P et al, “Plasma homocysteine after insulin infusion in type II diabetic patients with and without methionine intolerance”, Exp Clin Endocrinool Diabetes. January; 112(1): 4451, 2004.
Stanger O et al, “Consensus paper on the rational clinical use of homocysteine, folic acid and B-vitamins in cardiovascular and thrombotic diseases” Clin Chem Lab Med November; 41(11): 1392-1403, 2003
Geisel J et al, “The role of genetic factors in the development of hyperhomocysteinemia”, Clin Chem Lab Med, November; 41(11):427-34, 2003.
Chiang E P et al, “Abnormal vitamin B-6 status is associated with severity of symptoms in patients with rheumatoid arthritis”, American Journal of Medicine, March, 114(4) 283-7, 2003.
Guilland J C et al, “Hyperhomocysteinemia: an independent risk factor or a simple marker of vascutor disease” Pathol Biol March; 54(2): 101-10, 2003.
Cottington E M et al, “Adverse event associated with methionine loadng test, a case study”, Arteriosclerosis, Thrombosis and Vascular Biology; 22: 1046, 2002.
Cistemas M et al, “Cardiovascular risk factors in Chilean patients with rheumatoid arthritis” J Rheumatol August 29(8): 1619-22, 2002.
Rodriguez G et al, “Carotid atherosclerosis in patients with rheumatoid arthritis: a preliminary case-control study” Ann NY Acad Sci June; 966: 478-82 2002.
Desouza C et al, “Drugs affecting homocysteine metabolism: impact on cardiovascular risk”, Drugs, 62(4): 605-16, 2002.
Stulc T et al, “Folate supplementation prevents plasma homocysteine increases after fenofibrate therapy” Nutrition November-December; 17(11-12)973-74 2001.
Tice J A et al, “Cost effectiveness of vitamin therapy to lower plasma homocysteine levels for the prevention of coronary heart disease” JAMA 286(8); 936-43, 2001.
Schnyder G et al, “Decreased rate of coronary restenosis after lowering of plasma homocysteine levels” New England Journal Medicine November 29(345): 1593-1600, 2001.
Schulpis K H et al, “Elevated plasma homocysteine levels in patients on Isotretinoin therapy for cystic acne”, Int J Dermatol, January; 40(1): 33-6, 2001.
Apeland T et al, “Homocysteine concentrations and methionine loading in patients on antiepileptic drugs” Acta Neurol Scand April; 101(4): 217-23, 2000.
Zittoun & Zittoun, “Modern clinical testing strategies in cobalamin and folate deficiency” Semin Hematol January; 36(1): 35-46, 1999.
Fridman O “Hyperhomocysteinemia: atherothrombosis and neurotoxidcity” Acta Physiol Pharmacol Ther Latinoom 49(1): 21-30, 1999.
Lynn E G et al, “Homocysteine stimulates the production and secretion of cholesterol in hepatic cells” Biochem Biophys Acto August 28; 1393(2-3): 317-24, 2005.
Pema A F et al, “Metabolic consequences of folate-induced reduction of hyperhomocysteinemia in uremia” J Am Soc Nephrol December; 8(12): 1899-905, 1997.
Roubenoff R et al, “Abnormal homocysteine metabolism in rheumatoid arthritis” Arthritis Rheum. April; 40(4):718-22, 1997.
Sung J J et al, “Hyperhomocysteinoemia, helicobacter pylori, and coronary heart disease” Heart October; 76(4): 305-07, 1996.
den Heijer M et al, “Hyperhomocysteinemia as a risk factor for deep-vein thrombosis” New England Journal Medicine March 21; 334: 759-762, 1996.
Dudman N P et al, “Human homocysteine catabolism: three major pathways and their relevance to development of arterial occlusive disease” Journal Nutrition April; 126(4 suppl): 1295s-300s, 1996.
Linus Pauling Institute, Oregon State University, “The vascular toxicity of homocysteine and how to control it”
Kobayoshi, Yoichi et al, “Process for the production of methionine”, United States Patent Application No. 20050176115, Aug. 11, 2005.

I claim:

1. A method to limit homocysteine in a person being orally administered a prescription drug or over the counter drug, either of which contains at least one CH3 methyl group at one or more ends of the drugs chemical structure, in a single composition consisting of said drug and the antidote to homocysteine, which consists of at least one or more of the following: folic acid, vitamin b sub 12, vitamin b sub 6, trimethylglycine and choline.

2. The method of claim 1 wherein an antidote to homocysteine in the form of folic acid is present in the amount of at least 800 mcg per dose of the composition or in such an amount over 800 mcg per dose effective to limit homocysteine.

3. The method of claim 1 wherein an antidote to homocysteine in the form of vitamin b sub 12, preferably methylcobalamin, is present in the amount of at least 1000 mcg per dose of the composition or in such an amount over 1000 mcg per dose effective to limit homocysteine.

4. The method of claim 1 wherein an antidote to homocysteine in the form of vitamin b sub 6, preferably pyridoxyl 5 phosphate, is present in the amount of at least 100 mg per dose of the composition or in such an amount over 100 mg per dose effective to limit homocysteine.

5. The method of claim 1 wherein an antidote to homocysteine in the form of trimethylglycine is present in the amount of at least 500 mg per dose of the composition or in such an amount over 500 mg per dose effective to limit homocysteine.

6. The method of claim 1 wherein an antidote to homocysteine in the form of choline is present in the amount of at least 500 mg per dose of the composition or in such an amount over 500 mg per dose effective to limit homocysteine.
